(19) United States
(12) Patent Application Publication
(10) Pub. No.: US 2011/0301406 A1
(43) Pub. Date: Dec. 8, 2011

(54) ILLUMINATING APPARATUS AND CONTROL METHOD THEREOF

(75) Inventor: Toshihiro EHARA, Niiza-shi (JP)

(73) Assignee: Sanken Electric Co., Ltd., Niiza-shi (JP)

(21) Appl. No.: 13/115,380

(22) Filed: May 25, 2011

(30) Foreign Application Priority Data
Jun. 7, 2010 (JP) 2010-130144

(51) Int. Cl. A61M 21/00

(52) U.S. Cl. 600/27

(57) ABSTRACT

An illuminating apparatus 1 includes a light source 10 and a brightness controller 20. The brightness controller determines a fluctuation amplitude according to a 1/f power spectral model as a function of a fluctuation frequency that is selected to agree with one of human brain wave frequencies, sets the fluctuation amplitude as a brightness modulation amplitude, and modulates brightness of the light source by the brightness modulation amplitude at the fluctuation frequency.

### BACKGROUND OF THE INVENTION

1. Field of the Invention

The present invention relates to an illuminating apparatus that takes into account a human biological rhythm and a method of controlling such an illuminating apparatus.

2. Description of Related Art

Japanese Patent No. 2705364 discloses an illuminating apparatus that controls a light source in order to bring the user a comfortable feeling. A comfortable illuminating condition for a person, however, differs depending on circumstances. For example, an illuminating apparatus designed to relax the user is improper for a working environment in an office.

### SUMMARY OF THE INVENTION

The present invention provides an illuminating apparatus that takes into account a human biological rhythm and is capable of controlling illumination brightness depending on purposes, as well as a method of controlling such an illuminating apparatus.

According to an aspect of the present invention, the illuminating apparatus includes a light source and a brightness controller configured to determine based on a 1/f power spectral model a fluctuation amplitude for a fluctuation frequency that is selected to agree with one of human brain wave frequencies, set the fluctuation amplitude as a brightness modulation amplitude, and modulate brightness of the light source by the brightness modulation amplitude at the fluctuation frequency.

According to another aspect of the present invention, the method of controlling an illuminating apparatus includes setting a fluctuation frequency that is selected to agree with one of human brain wave frequencies, determining based on a 1/f power spectral model a fluctuation amplitude for the fluctuation frequency and setting the fluctuation amplitude as a brightness modulation amplitude, and modulating brightness of a light source of the illuminating apparatus by the brightness modulation amplitude at the fluctuation frequency.

### BRIEF DESCRIPTION OF THE DRAWINGS

FIG. 1 is a schematic view illustrating an illuminating apparatus according to a first embodiment of the present invention;

FIG. 2 is a 1/f power spectral model illustrating a relationship between fluctuation frequency f and fluctuation amplitude A in a logarithmic manner;

FIG. 3 is a view illustrating an example of modulating the brightness of a light source in the illuminating apparatus according to the first embodiment;

FIG. 4 is a schematic view illustrating an illuminating apparatus according to a second embodiment of the present invention; and

FIG. 5 is a schematic view illustrating an illuminating apparatus according to a modification of the second embodiment.

### DESCRIPTION OF PREFERRED EMBODIMENTS

The first and second embodiments of the present invention will be explained with reference to the drawings. Through the drawings, the same or like parts are represented with the same or like reference marks. The first and second embodiments exemplify apparatuses and methods that may realize a technical idea of the present invention. The technical idea of the present invention, however, is not restricted by these embodiments. The embodiments are modifiable in various ways without departing from the scope of the present invention defined in the claims.

### First Embodiment

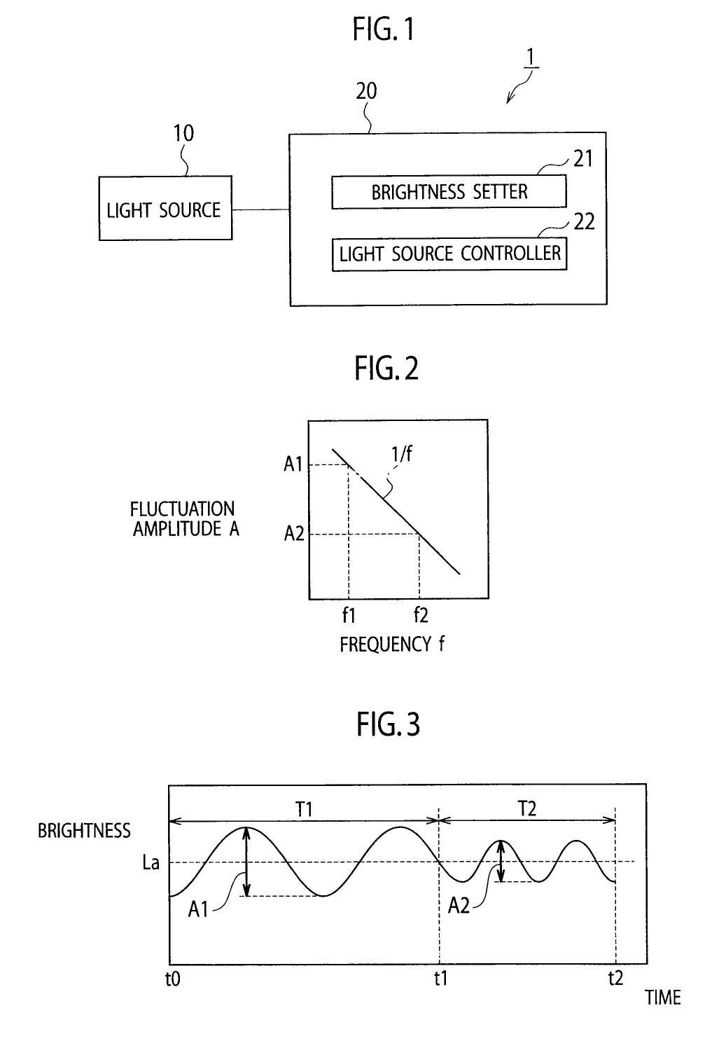

An illuminating apparatus according to the first embodiment of the present invention will be explained with reference to FIG. 1. The illuminating apparatus 1 includes a light source 10 and a brightness controller 20. The brightness controller 20 modulates the brightness of the light source 10 within a given brightness variation range at a given fluctuation frequency. The fluctuation frequency is selected to agree with one of human brain wave frequencies and the brightness variation range is a fluctuation amplitude that is determined based on a 1/f power spectral model as a function of the fluctuation frequency. The brightness controller 20 includes a brightness setter 21 and a light source controller 22. The brightness setter 21 sets a fluctuation amplitude (a power spectral density) as of a brightness according to a 1/f power spectral model as a function of a given fluctuation frequency. The light source controller 22 frequency-modulates brightness of the light source 10 at the fluctuation frequency and amplitude-modulates the same within the brightness variation range.

A human brain wave frequency has some relationship to a biological rhythm. For example, a delta wave is a brain wave of 1 to 3 Hz that occurs in a state of unconsciousness or deep sleep, a theta wave is a brain wave of 4 to 7 Hz that occurs in a state of meditation, inspiration, doze, or shallow sleep, an alpha wave is a brain wave of 8 to 13 Hz that occurs in a state of concentration, relax, or physical and psychological calmness, a beta wave is a brain wave of 14 to 30 Hz that occurs in a state of anxiety, tension, or daily living, and a gamma wave is a brain wave of 30 Hz or higher that occurs in a state of anger or excitement.

Apart from the above, there is a 1/f fluctuation that is a rhythm in the natural world to give a person with a comfortable feeling. FIG. 2 illustrates an example of a 1/f power spectral model with a double logarithmic expression. On the 1/f power spectral model, a frequency f and a fluctuation amplitude (power spectral density) A have an inverse proportion relationship, i.e., the higher the frequency f, the lower the fluctuation amplitude A. It is said that pleasant winds and exquisite music contain the 1/f fluctuation.

When receiving an external stimulus agreeing with a brain wave frequency, a person is led into a biological rhythm corresponding to the brain wave frequency. The illuminating apparatus 1 according to the present embodiment illuminates a person with light at a fluctuation frequency that is selected to agree with a brain wave frequency, to positively control a biological rhythm of the person. In addition, the illuminating apparatus 1 enhances the biological rhythm controlling effect by changing a brightness variation range (amplitude) of light according to the 1/f power spectral model.

The illuminating apparatus 1 sets an optional fluctuation frequency according to an user's purpose. The illuminating apparatus 1, therefore, is usable for any purpose or place. The illuminating apparatus 1 is installable in a room or outside a house, and depending on time or purpose, the brightness controller 20 controls brightness of the light source 10 at a given fluctuation frequency. For example, the brightness controller 20 controls the light source 10 so that brightness of the light source 10 fluctuates at a frequency in the range of delta wave (1 to 3 Hz) to theta wave (4 to 7 Hz) for sleep, in the range of alpha wave (8 to 13 Hz) to beta wave (14 to 30 Hz) for work and study, in the range of theta wave (4 to 7 Hz) to alpha wave (8 to 13 Hz) for relaxation, or in the range of beta wave (14 to 30 Hz) for wakeup.

A method of controlling brightness of the light source 10 in the illuminating apparatus 1 will be explained with reference to FIG. 3. In a period T1 from time t0 to t1, a fluctuation frequency for the light source 10 is set at f1, and in a period T2 from time t1 to t2, at f2. The frequency f1 is smaller than the frequency f2, and therefore, the frequency f1 is a minimum fluctuation frequency according to the example of FIG. 3.

An average brightness La is set for the light source 10. The average brightness La is optional and is set according to an environment such as home or office. The average brightness La is unchanged through the whole control period of brightness of the light source 10.

The brightness controller 20 finds on the 1/f power spectral model illustrated in FIG. 2 a fluctuation amplitude (brightness variation range) A1 for the fluctuation frequency f1 and a fluctuation amplitude A2 for the fluctuation frequency f2. More precisely, the fluctuation amplitude A1 for the minimum fluctuation frequency f1 is determined to be in the range of 1 to 25% of the average brightness La. This results in determining properties of the 1/f power spectral model illustrated in FIG. 2, so that the fluctuation amplitude A2 for the remaining fluctuation frequency f2 is determined relative to the fluctuation amplitude A1 for the fluctuation frequency f1.

The brightness controller 20 controls the light source 10 so that brightness of the light source 10 in the period T1 is modulated by the fluctuation amplitude (variation range) A1 at the fluctuation frequency f1, and in the period T2, modulated by the fluctuation amplitude A2 at the fluctuation frequency f2.

The brightness controller 20 may have an input unit that allows the user to set a fluctuation frequency selected for a specific purpose and a period in which brightness of the light source 10 is modulated at the fluctuation frequency. Instead, an external input unit may be used to send a command signal to the brightness controller 20, to set a fluctuation frequency and period in the brightness controller 20.

Periods for which fluctuation frequencies are set may be adjusted according to living hours. For example, in a period from early morning to evening during which a person is active, the light source 10 is modulated at a frequency selected from among the alpha wave frequencies of 8 to 13 Hz, and in a period from evening to sleep at a frequency selected from among the theta wave frequencies of 4 to 7 Hz to delta wave frequencies of 1 to 3 Hz. In a case for sleep or relaxation, the light source is modulated at a frequency selected from among the theta wave frequencies to delta wave frequencies according to a 1/f characteristics.

As mentioned above, each brain wave has a predetermined frequency range. A fluctuation frequency set for the light source 10 may be fixed within a given period, or may be changed in the period within the frequency range of a selected brain wave. For example, if the delta wave is selected to set a fluctuation frequency of the light source 10 in a given period, the fluctuation frequency may be changed within the frequency range of 1 to 3 Hz of the delta wave during the period. In this case, a fluctuation amplitude for the fluctuation frequency is also changed accordingly.

When the fluctuation frequency is modulated in relation with a given brain wave frequency range, it is preferable to change the fluctuation frequency gradually from low to high, and then, gradually from high to low, and periodically repeat the changing sequence. Changing the fluctuation frequency in such a way makes the user feel the brightness changes natural.

When put in the same environmental rhythm, i.e., the same fluctuation frequency for a long time, a person gradually feels no stimulus from the rhythm. Accordingly, changing the fluctuation frequency intermittently or continuously is effective to maintain the 1/f fluctuation effect of relaxation, daily living, sleep, or the like.

It is preferable to modulete brightness of the light source 10 in a manner of a sinusoidal wave. If the light source 10 is modulated according to a rectangular wave such as one provided by pulse width modulation (PWM), the light source 10 will cause flickering to give the user with an unpleasant feeling.

If a lower limit of brightness of the light source 10 is set to zero in controlling the fluctuation of the light source 10, a light-and-shade contrast of light from the light source 10 will be enhanced. In this case, even a fluctuation frequency conforming to a biological rhythm will cause a psychological unpleasantness when applied to the light source 10. To avoid this, the fluctuation amplitude A1 for the minimum fluctuation frequency f1 is set to be within the range of 1 to 25% of the average brightness La set for the light source 10. Then, brightness of the light source 10 is never zeroed even at the minimum fluctuation frequency f1 and the illuminating apparatus 1 causes no unpleasantness such as flickering.

Although the first embodiment employs two periods T1 and T2 for which different fluctuation frequencies f1 and f2 are set, one fluctuation frequency suitable for a specific purpose may be set to modulate brightness of the light source 10 only at the one fluctuation frequency. For example, if the illuminating apparatus 1 is used only for home relaxation, a fluctuation frequency selected from among the alpha wave frequencies is set to modulate brightness of the light source 10. In this case, a lowest one of the alpha wave frequencies is used as a minimum frequency to define properties of the 1/f power spectral model. The alpha wave has the frequency range of 8 to 13 Hz, and therefore, 8 Hz is the minimum fluctuation frequency to define properties of the 1/f power spectral model.

It is possible to set three or more periods having different fluctuation frequencies for the modulation of brightness of the light source 10. For example, a plurality of periods are set according to a human living rhythm and fluctuation frequencies are assigned to the plurality of periods, respectively. Setting a plurality of successive periods having different fluctuation frequencies may be automated after the user specifies an average brightness.

When different fluctuation frequencies are set for a plurality of periods, the brightness controller 20 determines a fluctuation amplitude for each of the fluctuation frequencies according to the 1/f power spectral model and employs the fluctuation amplitude as a brightness modulation amplitude for the period to which the fluctuation frequency is allocated. According to the fluctuation frequencies and brightness modulation amplitudes set for the plurality of periods, the brightness controller 20 modulates brightness of the light source 10 in each period. To determine the brightness modulation amplitudes for the plurality of periods, a minimum one of the fluctuation frequencies is used to fix properties of the 1/f power spectral model so that the brightness modulation amplitudes are determined according to the 1/f power spectral model in relation with the fluctuation frequencies.

For example, a fluctuation frequency selected from among the delta wave (1 to 3 Hz) to theta wave (4 to 7 Hz) frequencies and a corresponding fluctuation amplitude (brightness variation range) are set for a sleep period, a fluctuation frequency selected from among the alpha wave (8 to 13 Hz) to beta wave (14 to 30 Hz) frequencies and a corresponding fluctuation amplitude are set for a work and study period, a fluctuation frequency selected from among the theta wave (4 to 7 Hz) to alpha wave (8 to 13 Hz) frequencies and a corresponding fluctuation amplitude are set for a relaxation period, and a fluctuation frequency selected from among the beta wave (14 to 30 Hz) frequencies and a corresponding fluctuation amplitude are set for a wakeup period. These periods are properly combined according to a daily living rhythm and are used to control the light source 10.

In the example of FIG. 3, the brightness controller 20 controls the light source 10 so that the average brightness La of the light source 10 is constant through a plurality of periods having different fluctuation frequencies. Keeping the average brightness La constant without regard to the different fluctuation frequencies of the periods allows the user to selectively sense a 1/f fluctuation corresponding to the selected brain wave frequency. As mentioned above, the fluctuation amplitude of a minimum fluctuation frequency is set to be within the range of 1 to 25% of the average brightness La.

If the average brightness La is not constant and is variable, a variation in the average brightness La is superimposed on a 1/f fluctuation, to make the 1/f fluctuation not sensed by the user. The variation in the average brightness La not only causes the 1/f fluctuation not to be sensed by the user but also mixes, for example, the delta and alpha waves with each other because the variation is on a lower frequency side of the 1/f fluctuation. This will deteriorate, if occurs, the 1/f fluctuation effect of guiding the user to a specific biological rhythm.

To continuously change the frequency and modulation of brightness of the light source 10, the light source 10 must have excellent frequency characteristics and must keep chromaticity such as color temperature. When adjusting brightness of the light source 10 by controlling a drive current, the light source 10 is required to have a linear drive current-brightness relationship. Accordingly, the light source 10 may preferably be a light emitting diode (LED), an organic electroluminescence (EL) element, or the like but may not be a fluorescent lamp or a white bulb.

If the light source 10 is an LED or organic EL element having high color rendering properties, the illuminating apparatus 1 will realize high visibility with low power consumption.

As mentioned above, the illuminating apparatus 1 according to the present embodiment realizes a 1/f fluctuation by controlling the average brightness, minimum brightness, and brightness modulation amplitude of the light source 10.

In this way, the illuminating apparatus 1 according to the first embodiment sets a fluctuation frequency corresponding to one of the human brain wave frequencies, determines a brightness variation range for the fluctuation frequency on a 1/f power spectral model, and changes brightness of the light source 10 by the brightness modulation amplitude at the fluctuation frequency. Once the user selects and sets a brain wave frequency according to an arbitrary purpose, the illuminating apparatus 1 performs illumination to guide the user to a biological rhythm conforming to the purpose.

### Second Embodiment

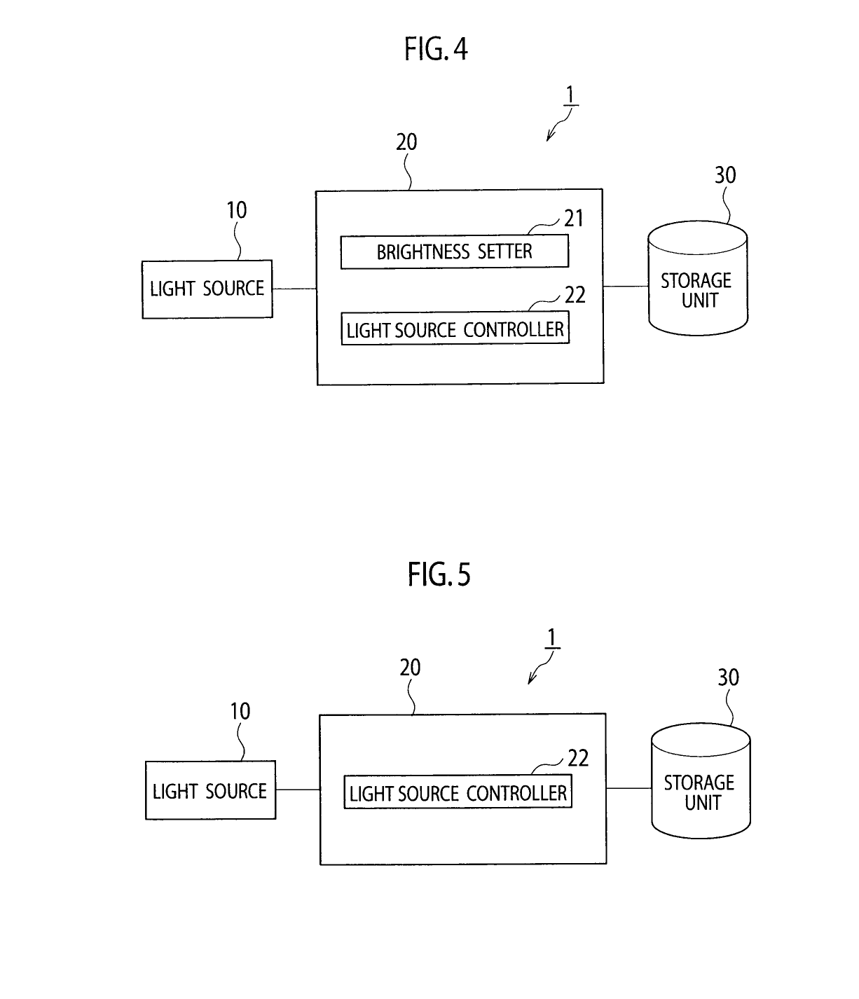

An illuminating apparatus 1 according to the second embodiment of the present invention will be explained with reference to FIG. 4. Compared with the illuminating apparatus 1 of the first embodiment illustrated in FIG. 1, the illuminating apparatus 1 of the second embodiment illustrated in FIG. 4 additionally has a storage unit 30 configured to store a plurality of fluctuation frequencies and corresponding brightness modulation ranges that are determined based on a 1/f power spectral model.

In FIG. 4, there is no need for a brightness controller 20 to find a brightness modulation range on the 1/f power spectral model every time when a fluctuation frequency is selected from among the brain wave frequencies.

The storage unit 30 stores a table of fluctuation frequencies and corresponding brightness variation ranges determined according to the 1/f power spectral model. Once the user sets a fluctuation frequency according to an arbitrary purpose, the brightness controller 20 retrieves from the storage unit 30 a fluctuation amplitude, i.e., a brightness modulation range corresponding to the fluctuation frequency. According to the fluctuation frequency and brightness variation range, the brightness controller 20 controls a light source 10. In this way, the present embodiment needs no determination of a brightness modulation range each time when a fluctuation frequency is selected, thereby saving operation time.

If a brightness modulation range for a fluctuation frequency set by the user is unavailable in the storage unit 30, a brightness setter 21 in the brightness controller 20 uses the 1/f power spectral model to find a brightness variation range for the set fluctuation frequency. The fluctuation frequency and corresponding brightness variation range at this time are stored in the table of the storage unit 30 for future use.

FIG. 5 illustrates an illuminating apparatus 1 according to a modification of the second embodiment. The modification is useful when no determination of a brightness variation range is expected. In this case, the brightness controller 20 needs no brightness setter 21, to make the illuminating apparatus 1 compact and energy saving.

The remaining part of the second embodiment and modification is the same as that of the first embodiment, and therefore, will not be explained.

Although the present invention has been described above by reference to certain embodiments of the present invention, the present invention is not limited to the embodiments described above. Modifications and variations of the embodiments described above will occur to those skilled in the art in light of the teachings.

Each of the illuminating apparatuses and methods of controlling them according to the present invention takes into account a human biological rhythm and achieves illumination at brightness appropriate for a given purpose.

This application claims benefit of priority under 35 USC §119 to Japanese Patent Application No. 2010-130144, filed on Jun. 7, 2010, the entire contents of which are incorporated by reference herein. The scope of the invention is defined with reference to the following claims.

What is claimed is:

1. An illuminating apparatus comprising:
a light source; and
a brightness controller configured to determine a fluctuation amplitude based on a 1/f power spectral model as a function of a fluctuation frequency that is related with human brainwave frequency, wherein the brightness controller sets the fluctuation amplitude as a brightness modulation amplitude, and modulates brightness of the light source by the brightness modulation amplitude at the fluctuation frequency.

2. The illuminating apparatus of claim 1, wherein the brightness controller includes:
a brightness setter configured to determine the fluctuation amplitude based on the 1/f power spectral model as a function of the fluctuation frequency and set the fluctuation amplitude as the brightness modulation range; and
a light source controller configured to modulate brightness of the light source according to the fluctuation frequency and brightness modulation amplitude.

3. The illuminating apparatus of claim 1, further comprising
a storage unit configured to store fluctuation frequencies and corresponding brightness modulation amplitudes, each of the stored brightness modulation amplitudes being determined based on the 1/f power spectral model in relation with one of the fluctuation frequencies.

4. The illuminating apparatus of claim 1, wherein
the fluctuation frequency is changed among a plurality of fluctuation frequencies that are set in advance in relation with a range of human brain wave frequencies.

5. The illuminating apparatus of claim 4, wherein
the following fluctuation frequency sequence of low-to-high and high-to-low is repeated:
(i) the fluctuation frequency is gradually changed from low to high among the plurality of fluctuation frequencies, and then
(ii) the fluctuation frequency is gradually changed from high to low in the same manner.

6. The illuminating apparatus of claim 1, wherein the brightness controller is configured to:
determine a fluctuation amplitude for each of a plurality of fluctuations frequencies that are set for a a plurality of periods according to the 1/f power spectral model, respectively;
set the fluctuation amplitude as a brightness modulation amplitude for the corresponding period; and
modulate brightness of the light source in each period according to the fluctuation frequencies and brightness modulation amplitude set for the plurality of periods.

7. The illuminating apparatus of claim 6, wherein:
an average brightness of the light source is fixed through the plurality of periods; and
the modulation amplitude determined for a lowest one of the fluctuation frequencies is set to be in the range of 1 to 25% of the average brightness.

8. The illuminating apparatus of claim 1, wherein
the light source is a light emitting diode or an organic electroluminescence element.

9. A method of controlling an illuminating apparatus, comprising:
setting a fluctuation frequency in relation with one of human brain wave frequencies;
determining a fluctuation amplitude according to a 1/f power spectral model as a function of the fluctuation frequency and setting the fluctuation amplitude as a brightness modulation amplitude; and
modulating brightness of a light source of the illuminating apparatus by the brightness modulation amplitude at the fluctuation frequency.

10. The method of claim 9, wherein
the fluctuation frequency is changed among a plurality of fluctuation frequencies that are set in advance according to a range of brain wave frequencies.

11. The method of claim 10, wherein:
the fluctuation frequency is gradually changed from low to high among the plurality of fluctuation frequencies;
the fluctuation frequency is gradually changed from high to low in the same manner; and
the fluctuation frequency changing sequence of low-to-high and high-to-low is periodically repeated.

12. The method of claim 9, wherein:
a plurality of fluctuation frequencies are set for a plurality of successive periods, respectively;
a fluctuation amplitude for each of the plurality of fluctuation frequencies is determined according to the 1/f power spectral model and is set as a brightness modulation amplitude for the corresponding period; and
brightness of the light source is modulated in each period according to the fluctuation frequencies and brightness modulation amplitudes that are set for the plurality of periods.

13. The method of claim 12, wherein
an average brightness of the light source is fixed through the plurality of periods.
